(19) United States
(12) Patent Application Publication
(10) Pub. No.: US 2025/0270489 A1
(43) Pub. Date: Aug. 28, 2025

(54) LEVELING SYSTEM FOR A BIOREACTOR SYSTEM

(71) Applicant: GLOBAL LIFE SCIENCES SOLUTIONS USA LLC, MARLBOROUGH, MA (US)

(72) Inventors: Fabio Ghidini, Hoegaarden (BE); Gerald Knight, Portsmouth (GB); Matthew Loveless, Portsmouth (GB)

(73) Assignee: GLOBAL LIFE SCIENCES SOLUTIONS USA LLC, MARLBOROUGH, MA (US)

(21) Appl. No.: 18/585,242

(22) Filed: Feb. 23, 2024

(51) Int. Cl. C12M 1/00; G01C 9/06

(52) U.S. Cl. CPC C12M 23/50; C12M 23/28; G01C 9/06

(57) ABSTRACT

An apparatus for levelling a bioprocessing device includes a load cell having a first portion and a second portion, the first portion being configured for attachment to a base surface. The apparatus further includes a levelling mechanism operatively connected to the second portion of the load cell, the second portion configured for attachment to a device surface, the device surface being in operative contact with a vessel of the bioprocessing device during use. The levelling mechanism is selectively adjustable to move the device surface relative to the base surface utilizing data from the load cell.

### BACKGROUND

### Technical Field

Embodiments of the invention relate generally to bioprocessing, and, more particularly, to an apparatus, system, and method that facilitates levelling of a bioprocessing device.

### Discussion Of Art

Mixers and bioreactors are often employed to carry out biochemical and biological processes and/or manipulate liquids and other products of such processes. Many bioreactors utilize closed, single use vessels, e.g., rigid, semi-rigid, or flexible containers and bags, that are fitted to or mounted on reusable equipment. Such vessels may also be referred to in the art as a single use systems (SUS). Such vessels may be in the form of rigid single use vessels, such as those used with fixed-bed bioreactors, as well as flexible bags that are housed within and are supported by an outer rigid structure such as a stainless-steel tank.

In fixed-bed reactors, the single use vessel is generally a rigid structure or housing that include a bed of carriers, e.g., polymeric fibers, which creates a large surface area for cells to adhere to. In use, the vessel may be fitted to an open platform or housing which may, for example, provide heat and include a drive unit for an impeller located withing the rigid single use vessel. Oxygenated culture medium is circulated through the bed to facilitate cell growth.

In certain fixed-bed systems, gas exchange inside the single use vessel is accomplished via a “waterfall,” which provides for optimal oxygenation and CO₂ removal. In particular, cell culture medium flows through the fixed bed from the bottom to the top of the bed.

At the top, the cell culture medium falls as a thin film down the outer wall where it takes up O₂ to maintain a high mass transfer coefficient in the bioreactor. As will be appreciated, levelling the single use reactor vessel facilitates an even waterfall which maximizes gas exchange within the reactor.

The dimensions of single use vessel, however, may vary somewhat from vessel to vessel due to stacked manufacturing tolerances, e.g., variance due to material shrinkage, etc. This may be particularly true with rigid single use vessels such as those used with fixed bed systems. Given these potential variances, floor levelling of the support structure to which a rigid single use vessel is fixed may not be ideal or sufficient. Indeed, precise vessel levelling may be desirable and/or necessary given potential vessel to vessel stack-up tolerances. Currently, levelling solutions do not provide a convenient, precise, and dynamic levelling mechanism to compensate for vessel-to-vessel variance.

In addition, vessel levelling, particularly levelling that involves altering the position of a rigid support structure relative to the floor beneath the structure involves manipulating levelling casters, which can be cumbersome. This process may involve the use of hand tools such as pliers and/or a wrench. Adjusting height may also involve dismounting the rigid support structure to add shims. As will be appreciated, it is generally desirable to have a levelling mechanism that may be easily hand manipulated without the use of tools or external materials.

Moreover, when fixed-bed reactors are transported, the platform or surface on which a single use rigid vessel is mounted during use is often fixed or locked for transportation in a raised or lowered transportation position. Raising/lowering and locking the platform/surface may involve tools and/or a separate locking mechanism.

In view of the above, there is a need for an apparatus, system, and method that facilitates precise and dynamic levelling of a bioprocessing device without the need for tools, shims, and the like to compensate for, e.g., stack-up manufacturing tolerances. There's also a need for a levelling mechanism that facilitates transportation of a bioprocessing device.

### Brief Description

Certain embodiments commensurate in scope with the originally claimed subject matter are summarized below. These embodiments are not intended to limit the scope of the claimed subject matter, but rather these embodiments are intended only to provide a brief summary of the possible embodiments. Indeed, the disclosure may encompass a variety of forms that may be similar to or different from the embodiments set forth below.

According to an aspect of the invention, an apparatus for levelling a bioprocessing device includes a load cell having a first portion and a second portion, the first portion being configured for attachment to a base surface. The apparatus further includes a levelling mechanism operatively connected to the second portion of the load cell, the second portion configured for attachment to a device surface, the device surface being in operative contact with a vessel of the bioprocessing device during use. Wherein the levelling mechanism is selectively adjustable to move the device surface relative to the base surface utilizing data from the load cell to achieve a substantially level vessel and/or a desired weight on the load cell.

In an embodiment, the load cell is a beam load cell.

In an embodiment, the base surface is a rigid structure of the bioprocessing device.

In an embodiment, the base surface may be a floor beneath the bioprocessing device.

In an embodiment, the levelling mechanism includes a rotatable wheel and a shaft operatively connected to the rotatable wheel and the load cell, the shaft having a threaded portion about which the rotatable wheel can be rotated. Wherein the rotatable wheel is in operative contact with the device surface such that rotation of the rotatable wheel about the shaft raises or lowers the device surface to facilitate levelling of the device surface.

In an embodiment, the device surface is a bottom surface of a housing of the bioprocessing device, the housing having an interior configured to facilitate bioprocessing of a fluid in the vessel.

In an embodiment, a portion of the shaft of the levelling mechanism extends into the interior of the housing, the portion including a stop that defines an upper bound on movement of the device surface relative to the base surface.

In an embodiment, the levelling mechanism further includes a threaded aperture in the shaft configured to receive a fastener, a base plate having an engagement portion configured to matingly engage a portion of the shaft, and a gasket between an attachment surface of the second portion of the load cell and the base plate. Wherein the second portion of the load cell has an aperture configured to allow passage of the fastener through the second portion, the fastener also passing through the gasket and base plate and into the threaded aperture in the shaft to secure the levelling mechanism to the load cell.

In an embodiment, rotation of the rotatable wheel by about 360 degrees raises or lowers the device surface by about 1 mm.

According to another aspect of the invention, a system for levelling a bioprocessing device includes a housing having an interior configured to facilitate bioprocessing of a fluid in a vessel of the bioprocessing device, the housing being in operative contact with the vessel during use of the bioprocessing device, the housing including a bottom surface, and a plurality of load cells each having a first portion and a second portion, the first portion attached to a base surface, each load cell having a levelling mechanism operatively connected to the second portion, the second portion being attached to the bottom surface of the housing. Wherein the levelling mechanism is selectively adjustable to move the bottom surface of the housing relative to the base surface utilizing data from the load cell to achieve a substantially level vessel and/or a desired weight on a load cell of the plurality of load cells.

In an embodiment, the bioprocessing device is a fixed bed bioreactor, and the plurality of load cells are three load cells.

In an embodiment, the bioprocessing device is a stirred tank bioreactor or mixer.

In an embodiment, the base surface is a rigid structure of the bioprocessing device.

In an embodiment, the base surface is a floor beneath the bioprocessing device.

In an embodiment, each levelling mechanism includes a rotatable wheel and a shaft operatively connected to the rotatable wheel and a load cell of the plurality of load cells, the shaft having a threaded portion about which the rotatable wheel can be rotated. Wherein the rotatable wheel is in operative contact with the bottom surface of the housing such that rotation of the rotatable wheel about the shaft raises or lowers the housing.

In an embodiment, a portion of the shaft of the levelling mechanism extends into the interior of the housing, the portion including a stop that defines an upper bound on movement of the housing relative to the base surface.

In an embodiment, each levelling mechanism further includes a threaded aperture in the shaft configured to receive a fastener, a base plate having an engagement portion configured to matingly engage a portion of the shaft, and a gasket between an attachment surface of the second portion of the load cell and the base plate. Wherein the second portion of the load cell has an aperture configured to allow passage of the fastener through the second portion, the fastener also passing through the gasket and base plate and into the threaded aperture in the shaft to secure the levelling mechanism to the load cell.

According to another aspect, a method of levelling a bioprocessing device includes receiving data from one or more load cells that are operatively connected to a housing of the bioprocessing device, the data indicative of a weight on a load cell of the one or more load cells, and raising or lowering a portion of the housing relative to a base surface using a levelling mechanism that is operatively connected to a load cell of the one or more load cells, until a load cell of the one or more load cells provides data indicating that the housing is level and/or a desired weight on a load cell of the one or more load cells has been achieved.

In an embodiment, raising or lowering a portion of the housing includes rotating a wheel about a threaded portion of a shaft, the shaft being operatively connected to the load cell. Wherein the wheel is in operative contact with a bottom surface of the housing such that rotation of the wheel about the shaft raises or lowers a portion of the housing.

### BRIEF DESCRIPTION OF THE FIGURES

The present invention will be better understood from reading the following description of non-limiting embodiments, with reference to the attached drawings, wherein below:

FIG. 1 is a perspective view of an exemplary bioprocessing device, e.g., a fixed bed bioreactor, suitable for use with embodiments of the invention;

FIG. 2 is a cut away view of a vessel, e.g., a single use fixed bed vessel, suitable for use with embodiments of the invention;

FIG. 3 is a perspective view of an apparatus for levelling a bioprocessing device in accordance with an embodiment of the invention;

FIG. 4 is an exploded perspective view of the apparatus of FIG. 3;

FIG. 5 is a bottom perspective view of multiple apparatus of FIG. 3 attached to a bottom surface of a housing of a bioprocessing device in accordance with an embodiment of the invention;

FIG. 6 is a cut away view of the housing of FIG. 5 depicting the attachment of the apparatus of FIG. 5 to the bottom surface of the housing as well as to a base surface of the bioprocessing device;

FIG. 7 is a perspective view of a shaft of a levelling apparatus according to an embodiment of the invention; and

FIG. 8 is a perspective view of a rotatable wheel of a levelling apparatus according to an embodiment of the invention.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts.

As used herein, the term “flexible” or “collapsible” refers to a structure or material that is pliable, or capable of being bent without breaking, and may also refer to a material that is compressible or expandable. An example of a flexible structure is a bag formed of polyethylene film.

A “vessel,” as the term is used herein, means a flexible bag, a flexible container, a semi-rigid container, or a rigid container, as the case may be. The term “vessel” as used herein is intended to encompass bioprocessing vessels having a wall or a portion of a wall that is flexible, single-use flexible bags, single-use rigid fixed bed containers, other containers or conduits commonly used in biological or chemical processing, including, for example, cell culture/purification systems, fermentation systems, mixing systems, media/buffer preparation systems, and filtration/purification systems.

Embodiments may be utilized in connection with a wide variety of biological and biochemical processes, which are referred to generally herein as “bioprocessing.” This term encompasses, but is not limited to, the various processes that occur in bioreactors, mixers, fermenters, and the like. A “bioprocessing vessel” is a vessel suitable for use with or in a bioreactor, mixer, fermenter, or other biological or chemical processing device. “Bioprocessing device” as used herein, includes bioreactor, mixer, fermenter, support structures, e.g., rigid tanks and the like, as well as a single-use reactor vessels, such as a rigid fixed-bed reactor vessel. Certain embodiments may be suitable for use in other industries where precise, easy to use levelling is desirable.

While embodiments are shown and described as for use with fixed-bed bioreactors, embodiments are not so limited. Certain embodiments may be suitable for use with stirred tank bioreactors and mixers as well as various other bioprocessing devices. Embodiments are likewise not limited to any specific type of viral vector or cell line.

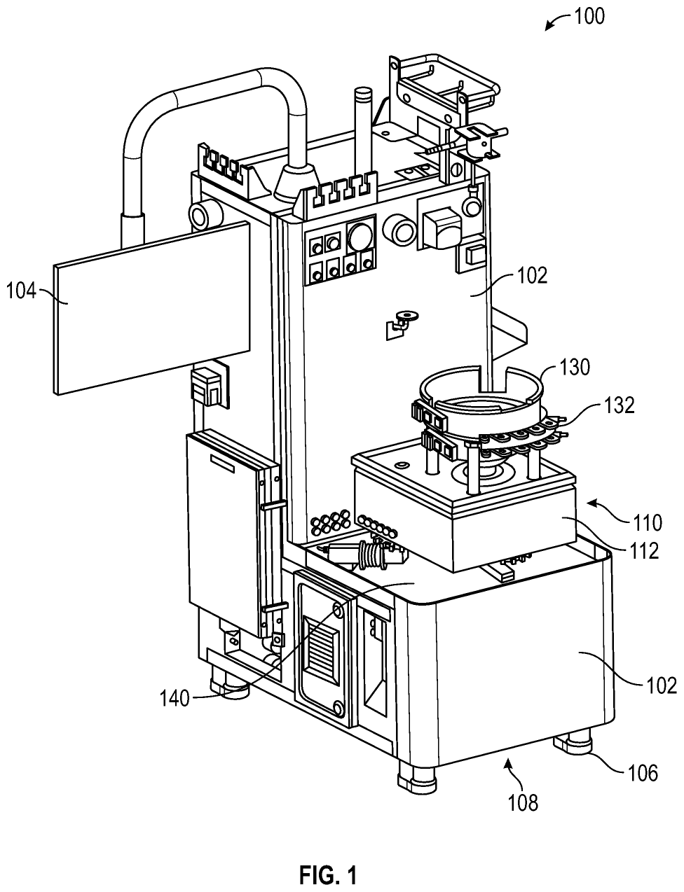

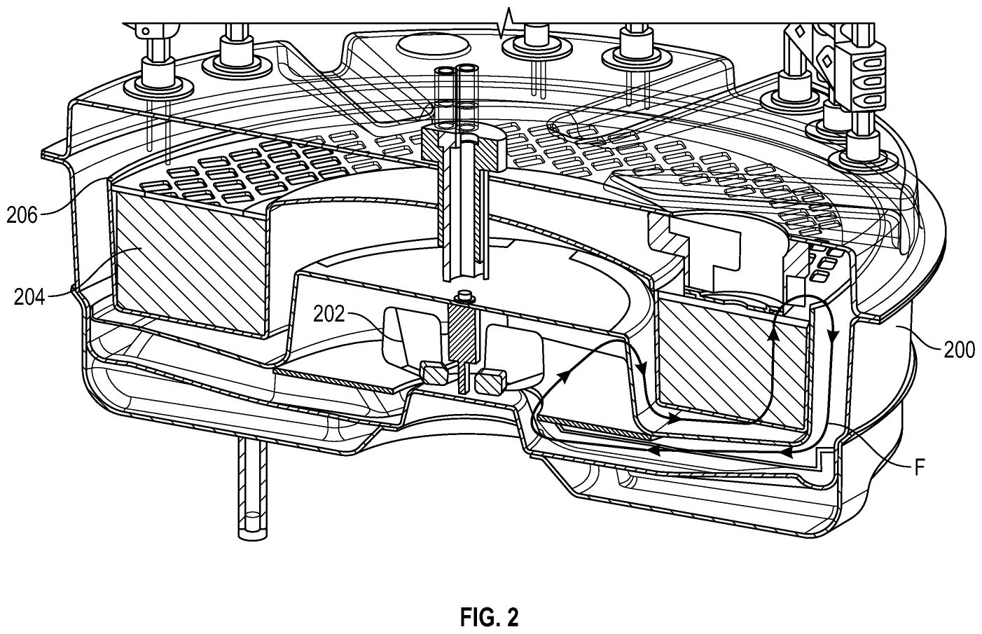

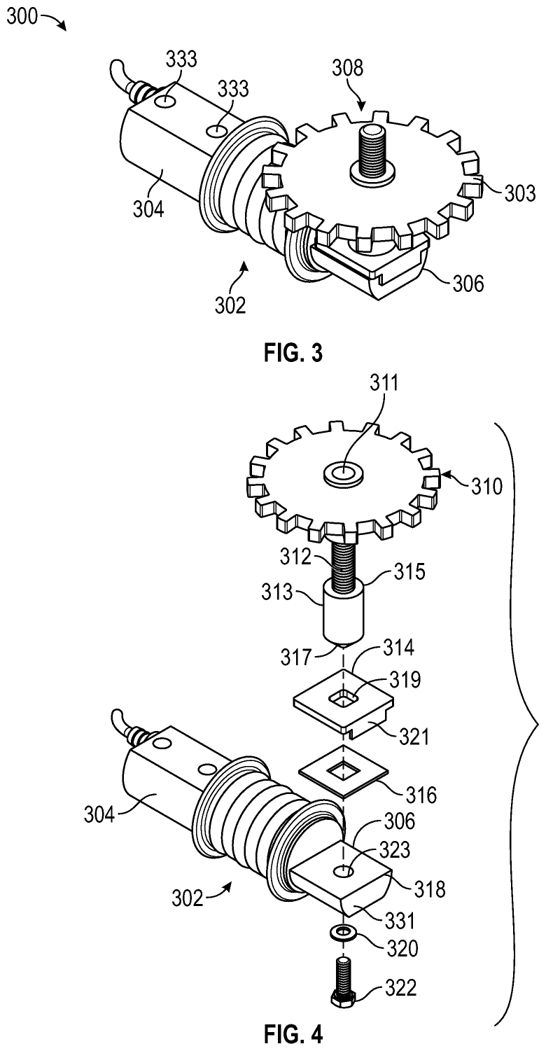

Referring now to FIG. 1, FIG. 2, FIG. 3 and FIG. 4, a bioprocessing device 100 suitable for use with embodiments of the present invention is depicted. The bioprocessing device 100 is a fixed-bed bioreactor that includes, among other components, a control assembly 102, which, as shown, is a substantially L-shaped, rigid cabinet. The control assembly 102 has an interior (not shown) that houses, for example, electrical panels, actuators, and fluid-flow/process automation related equipment. The control assembly 102 also includes a user interface 104 and a plurality of legs 106, which, as will be appreciated, contact the floor 108 beneath the bioprocessing device 100. In embodiments, the plurality of legs 106 may be equipped with lockable wheels with levelling feet and/or levelling casters to level the device relative to the floor.

The bioprocessing device 100 further includes an interface apparatus 110 which is configured to interface with a vessel 200, which is a single-use fixed bed vessel. The interface apparatus 110 includes a housing 112 having an interior 114 that is configured to facilitate bioprocessing of a fluid in the vessel 200. To that end, the interior 114 of the housing 112 has a drive motor 116, that is operatively connected to a gearbox 117, which is in turn operatively connected to a magnetic shaft/drive head 119, which is shown with a cap 121. In embodiments, the interior 114 of the housing 112 may be selectively opened and closed via a door and may be sealed via a gasket/seal. In use, drive head 119 is magnetically coupled to an impeller 202 within the single use vessel such that rotation of the drive head 119 rotates the impeller within the vessel to agitate fluid therein.

In an embodiment, the housing 112 is configured to receive and/or support a vessel support receptacle 130 that, in turn, is configured to receive the vessel 200. In particular, the vessel support receptacle 130 may provide heat to the vessel 200 in addition to receiving and/or supporting the vessel 200. In embodiments, the receptacle itself may be single use, and, to that end, may be manufactured from a rigid polymeric material. In other embodiments, the vessel support receptacle 130 may be integral to the vessel 200 or omitted. The housing 112 may further include a tubing management mechanism 132 which may be a U-shaped bracket having a plurality of teeth between which tubes and or valves may be placed.

As will be appreciated, while the depicted fixed bed bioreactor may be particularly suitable for embodiments of the invention, embodiments are not limited to any specific size or configuration of fixed bed reactor and may be used with larger and/or smaller reactors. Embodiments may be suitable for bench scale reactors as well as larger production scale models. Certain embodiments may be suitable for use with reactors having a fixed-bed volume of 0.2 to 25 L or more.

Turning back to FIGS. 1 and 2, in use, the vessel 200 is placed within the vessel support receptacle 130. Fluid input and output lines are attached and the vessel 200 is configured for fluid processing. Referring to FIG. 2, the vessel 200 includes a fixed bed 204, which creates a large surface area for cells to adhere to. Importantly, aerated culture media is circulated through the fixed bed 204 via the impeller 202. In particular, aerated media is circulated through the fixed bed 204 along a flow path F.

After passing through the fixed bed 204, the media overflows the outer circumferential wall 206 of the fixed bed 204 creating a waterfall-like falling film. This generates high rates of oxygen transfer and CO₂ stripping, allowing for the maintenance of high cell densities. As will be appreciated, it is desirable that the vessel 200 is level such that the waterfall is uniform around the outer circumferential wall 206 of the fixed bed 204 to maximize gas exchange within the vessel 200.

As previously mentioned, however, the dimensions of single use vessels may vary somewhat due to stacked manufacturing tolerances and floor levelling of the structure to which a single use vessel is attached may not be ideal or sufficient to compensate. In addition, vessel levelling, particularly levelling that involves altering the position of a rigid support structure relative to the floor beneath the structure, typically involves the use of hand tools such as pliers and/or a wrench and potentially the use of shims. As will be appreciated, it is generally desirable to have a precise levelling mechanism that may be easily hand manipulated without the use of any such tools or external materials to compensate for vessel-to-vessel variances.

Referring now to FIG. 3 and FIG. 4, an apparatus 300 for levelling a bioprocessing device is depicted. As shown, the apparatus 300 includes a load cell 302. The load cell 302 has a first portion 304 and a second portion 306. In the depicted embodiment, the first portion 304 and second portion 306 are at approximate distal ends of the load cell 302. The first portion 304 is configured for attachment to a base surface, such as a rigid structure 140 of the control assembly 102, or the floor 108 (FIG. 1).

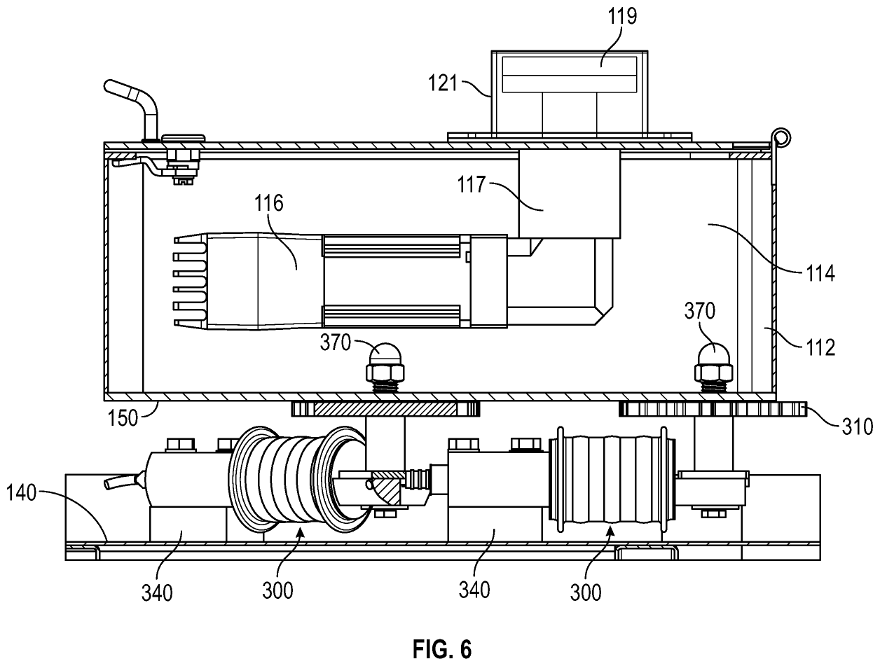

In embodiments, the first portion 304 has one or more apertures 333 which are configured to receive fasteners to attach the load cell 302 to the base surface. In the embodiment depicted in FIG. 6, the first portion 304 is secured via a block 340 to a rigid structure 140 (e.g., a metal panel) of the control assembly 102 of the bioprocessing device 100. In other embodiments, the second portion 306 may be connected to a base surface using other attachment mechanisms.

The apparatus 300 further includes a levelling mechanism 308 which is operatively connected to the second portion 306 of the load cell 302. The second portion 306 of the load cell 302 is configured for attachment to a bioprocessing device surface, e.g., a bottom surface 150 of the housing 112. The device surface being in operative contact with a vessel (e.g., vessel 200) of the bioprocessing device during use.

The levelling mechanism 308 is selectively adjustable to move the device surface relative to the base surface to level the device surface to achieve a substantially level vessel and/or a desired weight on the load cell. In particular, the apparatus 300 may utilize data from the load cell that is indicative of a level position of the device surface, and/or data indicative of a desired weight on the load cell, e.g., a percentage of a total weight or a specific weight. In embodiments, the load cell is a digital beam load cell, for example, an Eilersen BL-Ex load cell. However, the invention is not limited to a specific type of load cell or weighing mechanism.

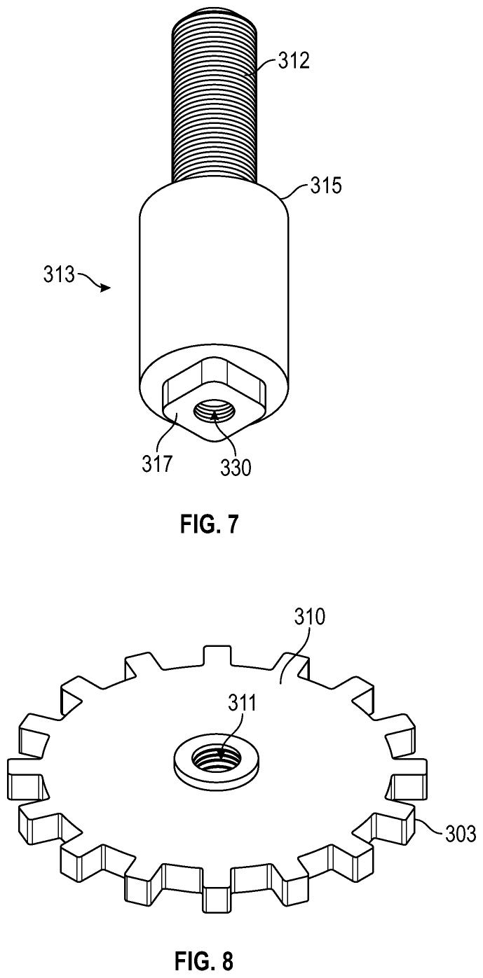

Referring to FIG. 4, FIG. 7, and FIG. 8, in an embodiment, the levelling mechanism 308 includes a rotatable wheel 310 that has a threaded hole 311 that is configured to receive a threaded portion 312 of a shaft 313 such that the wheel can be rotated about the shaft 313. As described in greater detail below, rotating the wheel about the shaft raises or lowers the bioprocessing device surface (e.g., the bottom surface 150 of the housing 112) to facilitate levelling of the device surface and a vessel 200 attached thereto. The shaft 313 has a shoulder portion 315 which limits the travel (i.e., forms a lower bound or stop) of the rotatable wheel 310 on the shaft 313.

As shown, the rotatable wheel 310 has a series of teeth 303 which extend around the circumference of the wheel. The series of teeth 303 are configured to allow a user to easily manipulate and rotate the rotatable wheel 310. As such, the space between teeth 303 may be selected based on ergonomic factors such as hand and finger size. As will be appreciated, the size, number, and placement of the teeth 303 may vary and the invention is not limited in this regard. In certain embodiments, the rotatable wheel 310 may have a relatively small number of teeth equally spaced about the circumference of the rotatable wheel 310. In certain embodiments, the teeth 303 may be textured, e.g., knurled, to increase grip. The teeth 303 also need not be a specific shape and squared, rounded, or other shaped teeth may be employed.

In an embodiment, the levelling mechanism 308 is secured to the load cell 302 via a threaded fastener 322 that extends through an aperture 323 in the second portion 306 of the load cell 302. The threaded fastener 322 extends into a threaded bore 330 in a bottom portion of the shaft 313 and may be attached via a washer 320 (FIG. 4 and FIG. 7).

The bottom portion of the shaft 313 further includes a protrusion 317 that is sized and shaped to fit into and matingly engage an engagement portion 319 of a base plate 314. In an embodiment, the engagement portion 319 is a rectangular aperture (and the protrusion 317 has a corresponding rectangular shape) though other aperture shapes (and engagement portions) may be employed. The base plate 314 includes a lip 321 that is configured to abuttingly contact a front surface 331 of the second portion 306 of the load cell 302. The lip 321, along with the protrusion 317 and engagement portion 319, functions to prevent rotation of the shaft 313 while the wheel is rotated. In an embodiment, the base plate 314 sits on top of a gasket 316, which is configured to provide shock absorption and noise reduction to the load cell.

In embodiments, with the exception of gasket 316, the components of the levelling mechanism 308, e.g., the shaft 313 and rotatable wheel 310, are manufactured from a rigid material such as a metal. In specific embodiments, the components are manufactured from a chromium-nickel based steel, e.g., SS316. In other embodiments, a polymeric material may be employed to the extent that such material has properties sufficient for the intended functionality of the invention.

As will be appreciated, the load cells 302 may be operatively connected to a controller of the bioprocessing device. The connection may be wired or wireless, and the load cells 302 may be provide readings to the controller. In other embodiments each load cell itself may have a user interface, screen, or the like that can provide data to a user regarding a load on the load cell.

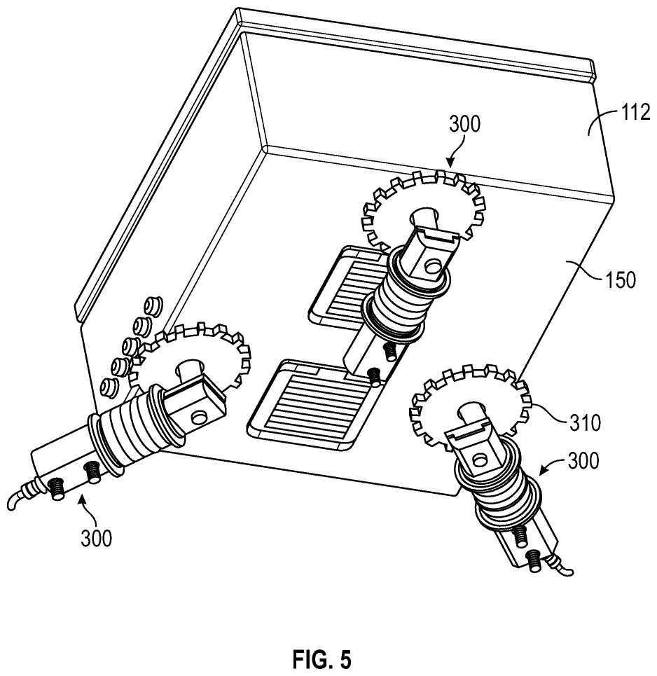

Referring now to FIG. 5 and FIG. 6, in an embodiment, a system for levelling a bioprocessing device includes three levelling apparatus that are attached to a bottom surface 150 of the housing 112. As will be appreciated, in other embodiments, there may be greater or fewer than three levelling apparatus. For example, in a specific embodiment, there may be an apparatus 300 under each of the four corners of the housing or similar rectangular structure/device surface.

Each of the three levelling apparatus are attached to the base surface, e.g., the rigid structure 140 of the control assembly 102, via block 340, and to the device surface, e.g., bottom surface 150 of the housing 112. In particular, the bottom surface 150 of the housing 112 contains apertures that allow for the passage of at least a portion of the threaded portion 312 of the shaft 313 into the interior 114 of the housing 112. The portion of the shaft 313 that extends into the housing is topped or capped with a threaded dome nut 370. The threaded dome nut 370 defines an upper bound on movement of the device surface relative to the base surface, i.e., how high the levelling apparatus can raise the device surface relative to the base surface. The shoulder portion 315 of the shaft 313 provides a lower limit or stop on how low the levelling apparatus can lower the device surface relative to the base surface.

In use, the bottom surface 150 of the housing 112 is in contact with, e.g., rests on, the rotatable wheel 310. Rotation of the rotatable wheel 310 about the threaded portion 312 of the shaft 313 causes the wheel to raise or lower causing the housing 112 to raise or lower via bottom surface 150.

As will be appreciated, 360 degrees of rotation of the rotatable wheel 310 lifts or drops a corner of the housing 112 by one thread pitch. This allows for precise tuning as, for example, in an embodiment, 360° rotation of the wheel results in a 1 mm change in position and a 45° rotation results in a 0.125 mm change in position. In other embodiments, the thread pitch may vary somewhat, and embodiments of the invention are not intended to be limited to any specific pitch measurement, though fine pitch measurements may be desirable.

In embodiments, the length of the path of travel of the rotatable wheel on the shaft 313 may vary and the distance the device surface may be raised or lowered relative to the base surface may vary. Thus, it may be possible to adapt the apparatus 300 to different levelling applications by varying the length of the threaded portion 312 of the shaft 313. In a specific embodiment, the threaded portion has a length of about 38 mm and the shaft 313 has an overall length of about 78 mm.

Embodiments of the invention also contemplate a method of levelling a bioprocessing device. The method includes receiving data from one or more load cells that are operatively connected to a housing of the bioprocessing device. A user will then raise or lower a portion of the housing relative to a base surface using a levelling mechanism that is operatively connected to a load cell of the one or more load cells, until at least one load cell of the one or more load cells provides data indicating that the housing is level and/or that the weight on the at least one load cell is at a desired level.

In embodiments, raising or lowering a portion of the housing involves rotating a wheel about a threaded portion of a shaft, the shaft being operatively connected to the load cell. The wheel is in operative contact with a bottom surface of the housing such that rotation of the wheel about the shaft raises or lowers a portion of the housing.

In embodiments, the data from the load cells may be expressed as the percentage of weight on an individual load cells as compared to the total weight on all load cells deployed. That is, with three load cells, each should have 33% of the overall load. Thus, the load cell may provide a reading that indicates a percentage of weight. The levelling mechanisms may then be utilized to increase or decrease the percentage of weight. The load cells may also provide a specific weight (e.g., grams, kilograms, etc.) on each load cell.

In certain embodiments, the desired weight on each load cell may not be equal and the desired position or state of the vessel and/or housing may not be a substantially level position. In this regard, embodiments are useful for fine tuning weight distributions for a variety of purposes and not just for levelling.

Embodiments of the invention also provide an ease of transporting a bioprocessing device for shipment and installation. For example, it is desirable to fix device components relative to one another to reduce the possibility of damage during transportation. Embodiments allow for each levelling apparatus to be raised to maximum height, e.g., where the interior of the bottom surface of the housing contacts the upper bound or stop (e.g., the nuts) on each shaft. In this position, there is no play and the device is fixed. Placing the housing in this position, and removing it for use, is quicky and easily accomplished via embodiments of the inventive levelling apparatus.

While the embodiment contemplates manual adjustment of the levelling mechanism via the rotatable wheel, in other embodiments the levelling mechanism may utilize a motor, e.g., a linear actuator or the like. Certain embodiments, may utilize continuous data from the load cells to make automated small real time or near real time levelling adjustments.

In an alternative embodiment that is not depicted, levelling apparatus of may be secured to legs or a base portion of a stirred tank (or other type) reactor. That is, each leg of the rigid tank that, for example, receives and supports a single use vessel/bag, may receive a levelling apparatus. In such embodiments, the apparatus may be used to level the device surface of the tank relative to a base surface, e.g., the floor.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

While the dimensions and types of materials described herein are intended to define the parameters of the invention, they are by no means limiting and are exemplary embodiments. Many other embodiments will be apparent to those of skill in the art upon reviewing the above description.

The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.”

Moreover, in the following claims, terms such as “first,” “second,” “upper,” “lower,” “bottom,” “top,” etc. are used merely as labels, and are not intended to impose numerical or positional requirements on their objects. Further, the limitations of the following claims are not written in means-plus-function format and are not intended to be interpreted as such, unless and until such claim limitations expressly use the phrase “means for” followed by a statement of function void of further structure.

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal language of the claims.

1. An apparatus for levelling a bioprocessing device comprising:
a load cell having a first portion and a second portion, the first portion being configured for attachment to a base surface; and
a levelling mechanism operatively connected to the second portion of the load cell, the second portion configured for attachment to a device surface, the device surface being in operative contact with a vessel of the bioprocessing device during use;
wherein the levelling mechanism is selectively adjustable to move the device surface relative to the base surface, utilizing data from the load cell, to achieve a substantially level vessel and/or a desired weight on the load cell.

2. The apparatus of claim 1 wherein load cell is a beam load cell.

3. The apparatus of claim 1 wherein the base surface is a rigid structure of the bioprocessing device.

4. The apparatus of claim 1 wherein the base surface is a floor beneath the bioprocessing device.

5. The apparatus of claim 1 wherein the levelling mechanism comprises:
a rotatable wheel; and
a shaft operatively connected to the rotatable wheel and the load cell, the shaft having a threaded portion about which the rotatable wheel can be rotated;
wherein the rotatable wheel is in operative contact with the device surface such that rotation of the rotatable wheel about the shaft raises or lowers the device surface to facilitate levelling of the device surface.

6. The apparatus of claim 5 wherein the device surface is a bottom surface of a housing of the bioprocessing device, the housing having an interior configured to facilitate bioprocessing of a fluid in the vessel.

7. The apparatus of claim 6 wherein a portion of the shaft of the levelling mechanism extends into the interior of the housing, the portion including a stop that defines an upper bound on movement of the device surface relative to the base surface.

8. The apparatus of claim 5 wherein the levelling mechanism further comprises:
a threaded aperture in the shaft configured to receive a fastener;
a base plate having an engagement portion configured to matingly engage a portion of the shaft; and
a gasket between an attachment surface of the second portion of the load cell and the base plate;
wherein the second portion of the load cell has an aperture configured to allow passage of the fastener through the second portion, the fastener also passing through the gasket and base plate and into the threaded aperture in the shaft to secure the levelling mechanism to the load cell.

9. The apparatus of claim 5 wherein rotation of the rotatable wheel by about 360 degrees raises or lowers the device surface by about 1 mm.

10. A system for levelling a bioprocessing device comprising:
a housing having an interior configured to facilitate bioprocessing of a fluid in a vessel of the bioprocessing device, the housing being in operative contact with the vessel during use of the bioprocessing device, the housing including a bottom surface; and
a plurality of load cells each having a first portion and a second portion, the first portion attached to a base surface, each load cell having a levelling mechanism operatively connected to the second portion, the second portion being attached to the bottom surface of the housing;
wherein the levelling mechanism is selectively adjustable to move the bottom surface of the housing relative to the base surface utilizing data from at least one load cell of the plurality of load cells to achieve a substantially level vessel position or to achieve a desired weight on a load cell of the plurality of load cells.

11. The system of claim 10 wherein the bioprocessing device is a fixed bed bioreactor, and the plurality of load cells are three load cells.

12. The system of claim 10 wherein the bioprocessing device is a stirred tank bioreactor or mixer.

13. The system of claim 10 wherein the base surface is a rigid structure of the bioprocessing device.

14. The system of claim 10 wherein the base surface is a floor beneath the bioprocessing device.

15. The system of claim 10 wherein each levelling mechanism comprises:
a rotatable wheel; and
a shaft operatively connected to the rotatable wheel and a load cell of the plurality of load cells, the shaft having a threaded portion about which the rotatable wheel can be rotated;
wherein the rotatable wheel is in operative contact with the bottom surface of the housing such that rotation of the rotatable wheel about the shaft raises or lowers the housing.

16. The system of claim 15 wherein a portion of the shaft of the levelling mechanism extends into the interior of the housing, the portion including a stop that defines an upper bound on movement of the housing relative to the base surface.

17. The system of claim 15 wherein each levelling mechanism further comprises:
a threaded aperture in the shaft configured to receive a fastener;
a base plate having an engagement portion configured to matingly engage a portion of the shaft; and
a gasket between an attachment surface of the second portion of the load cell and the base plate;
wherein the second portion of the load cell has an aperture configured to allow passage of the fastener through the second portion, the fastener also passing through the gasket and base plate and into the threaded aperture in the shaft to secure the levelling mechanism to the load cell.

18. A method of levelling a bioprocessing device comprising:
receiving data from one or more load cells that are operatively connected to a housing of the bioprocessing device, the data indicative of a weight on a load cell of the one or more load cells; and
raising or lowering a portion of the housing relative to a base surface using a levelling mechanism that is operatively connected to a load cell of the one or more load cells, until a load cell of the one or more load cells provides data indicating that the housing is level and/or a desired weight on a load cell of the one or more load cells has been achieved.

19. The method of claim 18 wherein raising or lowering a portion of the housing comprises:
rotating a wheel about a threaded portion of a shaft, the shaft being operatively connected to the load cell;
wherein the wheel is in operative contact with a bottom surface of the housing such that rotation of the wheel about the shaft raises or lowers a portion of the housing.
